(19) United States
(12) Patent Application Publication
(10) Pub. No.: US 2002/0111673 A1
(43) Pub. Date: Aug. 15, 2002

(54) Light activated composite stents and vascular prosthetics

(76) Inventors: Carvel Holton, Blacksburg, VA (US); Brian Love, Blacksburg, VA (US); Andre A. Muelenaer, Roanoke, VA (US); Patricia Dolez, Blacksburg, VA (US)

Correspondence Address: Whitham, Curtis & Christofferson, PC, 11491 Sunset Hills Road - #340, Reston, VA, 20190, US

(21) Appl. No.: 10/014,811

(22) Filed: Dec. 14, 2001

Related U.S. Application Data
(60) Provisional application No. 60/255,074, filed on Dec. 14, 2000.

(51) Int. Cl. A61F 2/06

(52) U.S. Cl. 623/1.21

(57) ABSTRACT

The invention provides tailored, biocompatible conduits for use as, for example, stents, vascular grafts, and drug delivery devices, and methods of making the conduits. The conduits are formed from a resin-impregnated matrix that is introduced to a site of interest in a malleable state, formed into a desired shape, and cured by exposure to radiant energy. The radiant energy may be visible light.

This application claims priority to United States provisional application serial number 60/255,074, the complete contents of which are herein incorporated by reference.

### DESCRIPTION

### BACKGROUND OF THE INVENTION

1. Field of the Invention

The invention generally relates to tailored stents and vascular prostheses. In particular, the invention provides tailored stents and vascular prostheses comprised of light activated composites that are cured in situ.

2. Background of the Invention

A stent is a medical device used in the tubular passage ways of the body to maintain an open lumen. For example, stent placement is routinely used to treat coronary occlusions in conjunction with angioplasty procedures. A stent placed within a blood vessel serves to act as “scaffolding”, maintaining vessel diameter and thereby allowing increased blood flow.

Stents are typically formed of rigid wire or plastic mesh material. They are inserted directly into a damaged area of a blood vessel after the vessel is expanded by angioplasty. The stent itself is inserted in a collapsed form and is expanded after placement by balloon catheter. Stents are available in several standard sizes which vary in length and diameter. Unfortunately, they are not infinitely variable and there is currently no way to insure that the fit of a stent to a damaged vessel will be precise. As a result, problems may occur after placement of the stent, including collapse of the stent after placement, problems with restenosis within the stent itself, and erosion of the metallic stent through the vessel wall. Further, conventional stents are straight and not adaptable to the natural curvatures exhibited by blood vessels in the heart. In addition, there are no devices currently available that provide continuous stenting in a “Y” configuration.

Because current stents are metallic, their implementation must be monitored with x-rays. It would be preferable to observe stents using magnetic resonance imaging (MRI) since MRI is associated with fewer health risks than are x-rays. However, metallic stents may be affected by MRI energy (producing heating and tissue damage) and also may produce artifacts when tissues are viewed by this modality. This limits the ability to utilize MRI angiography for follow-up of angioplasty and stent placement. Thus, there is currently a desire to switch to non-metallic stents, which would allow MRI imaging to be used to monitor their implementation. MRI technology is safer for human tissues and easier for doctors to use.

Similarly, as technological efforts improve the ability to produce prosthetic blood vessels of almost matching properties [1], as well as artificially grown vascular grafts [2, 3], a problem remains for abnormal shaped blood vessels that require tailorable, on-site designed grafts [4, 5, 6].

It would be highly desirable to have available prosthetic blood vessels and stents that are tailored to fit the vessel(s) where they function. Preferably, the devices would be pliable yet rigid and formed from biocompatible and non-thrombogenic materials. Further, it would be especially desirable if the material of which the vessels and stents are formed could be safely monitored via MRI techniques.

### SUMMARY OF THE INVENTION

It is an object of this invention to provide tailored biocompatible conduits for use in such applications as stents and vascular prostheses. The tailored conduits are fabricated from resin-matrix composites in which the resin is cured (polymerized) by exposure to selected wavelengths of radiant energy. The uncured composite is formed to a desired size and shape at the site of use (e.g. within or adjoining a blood vessel) and may be made to conform to the natural contours of the site. The composite is then cured rapidly by exposure to an appropriate wavelength of radiant energy. In a preferred embodiment, the radiant energy is in the visible range.

Advantages of the tailored conduits of the present invention include a high level of biocompatibility, (including a smooth surface and a rigid yet pliable consistency approximating that of natural tissue), the ability to individually tailor the conduits to fit the site where they will function, a very short cure time, and the possibility of relatively large cured composite thickness due to the depth of penetration of visible light (compared to, for example, ultraviolet light).

The invention also provides a drug delivery device in that pharmacologically active agents may be impregnated in or attached to the composites.

### BRIEF DESCRIPTION OF THE DRAWINGS

FIG. 1A-C. Schematic representation of the fabrication of a stent of the present invention. 1=uncured resin-impregnated matrix; 2=vessel; 3=occluded region of vessel; 4=catheter; 5=balloon; 6=visible light source; 10=cured resin-impregnated matrix.

FIG. 2. Typical elastic modulus curve for fiberglass composite (thin fiberglass, basic resin, 1 minute cure). X axis represents displacement (mm) and Y axis represents force (N).

FIG. 3. Typical elastic modulus curve for nylon composite (basic resin, 2 minute cure). X axis represents displacement (mm) and Y axis represents force (N). Arrow A indicates fracture of the resin in between the fibers. Arrow B indicates successive fracture of the fibers.

FIG. 4. Elastic modulus results. ▪=thick fiberglass+basic resin; ●=thin fiberglass+basic resin; ▴=nylon+basic resin; ▾=thick fiberglass+plasticized resin. X axis is cure time (minutes). Y axis is elastic modulus (kPa).

FIG. 5. Typical flexural modulus curve for thin fiberglass (basic resin, 1 minute cure) tube composite. X axis is displacement (mm); Y axis is force (N).

FIG. 6. Results of flexural modulus studies with thick and thin fiberglass using basic and plasticized resin. Y axis is flexural modulus (kPa).

FIG. 7. Adhesion strength values of bonds made underwater on various types of substrate materials using alkoxylated cyclohexane dimethanol diacrylate based resin. Y axis is bonding strength (Mpa).

### DETAILED DESCRIPTION OF THE PREFERRED EMBODIMENTS OF THE INVENTION

The present invention provides tailored artificial conduits that are biocompatible. In preferred embodiments, the conduits function as stents and prosthetic vessels for use in, for example, vascular grafts. They are “tailored” in that they are made from material that can be molded to conform to a desired shape at the site where they are to function, and then cured in place at the site. The cured conduit maintains a rigid yet somewhat pliable consistency. The conduits of the present invention can each be individually adapted to fit a physiological location, for example within or adjoined to abnormal, injured or transplanted vessels. As a result, use of the conduits of the present invention in, for example, vascular grafts results in less pulsatile blood leakage and less deformation of the blood vessel. Use of the conduits of the present invention as stents provides greater stability when compared to conventional stents. Further, the smooth surface of such stents is less likely to cause clot formation and inflammatory responses than wire mesh materials of conventional stents.

The tailored conduits of the present invention are made from matrix material or mesh impregnated with an adhesive or polymeric resin that is photopolymerized by exposure to radiant energy such as visible light. Resins of this type are in liquid state until exposed to the particular wavelength(s) of e.g. visible light to which they are sensitive. Such exposure causes polymerization and hardening of the resin. When matrix material is impregnated with such a resin, the entire composite becomes solidified under light exposure. Advantages of using material that is cured on exposure to light include that they are “cured on demand” by exposure to directed illumination, e.g. in vivo after positioning of the uncured material at an appropriate site of interest. The other major advantage is the fast speed of the polymerization reaction, less than one minute. In addition, utilizing resins that cure in response to visible light in particular, (as opposed to those that cure in response to ultraviolet light), allows them to harden in a physiological setting without exposing tissue to potentially damaging UV light. Further, visible light penetrates deeper than UV light, allowing resins of this type to deliver an adhesive cure of significantly greater thickness than UV-activated resins, offering greater stability of the conduit. Finally, these resins cure with a very moderate release of heat, which limits the risk of tissue damage. However, it will be understood that for some applications, UV light may also be utilized.

The resins utilized in the practice of the present invention are cured by a free radical mechanism and comprise at least three components: a principal monomer (e.g. bis-phenol A diglycidyl methacrylate, bis-GMA), a viscosity modifier, and a photoinitiator. An activator may also be required (as an intermediary to create free radicals from a product of the photoinitiator) if the photoinitiator itself is not capable of forming free radicals directly upon exposure to light. Those of skill in the art will recognize that many suitable combinations of monomers and photoinitiators (with or without an activator, as appropriate) exist which may be utilized in the practice of the present invention. Examples include but are not limited to: principal monomers, bis-phenol A diglycidyl methacrylate and other acrylate monomers; viscosity modifiers, triethylene glycol dimethacrylate, alkoxylated cyclohexane dimethanol diacrylate and the other diacrylate monomers; photoinitiators, camphorquinone, ketones, thioxanthone and 3-ketocoumarins; and activators, N, N dimethyl-p-toluidine, amines and tertiary amines. Further, those of skill in the art will recognize that other suitable agents may also be added to the resin, examples of which include but are not limited to antioxidants, plasticizers, fillers, and the like. In a preferred embodiment, the resin for a stent and for a vascular graft will be made of bisphenol A diglycidyl methacrylate, triethylene glycol dimethacrylate, N, N dimethyl-p-toluidine, and camphorquinone.

Those of skill in the art will recognize that many suitable matrix materials exist that may be employed in the practice of the present invention. Examples of such materials include but are not limited to various flexible, open weave, loose braid or knit tubular fibers and woven, knitted and non-textured fabrics made from e.g. fiberglass, nylon, polyester, polyurethanes, polytetrafluoroethylene, cotton, silk, and the like. The matrix material may be in any appropriate shape depending on the intended use. For example, the matrix may resemble fabric strips, tubes, etc. of any desired overall shape and dimensions as suitable for the particular use. In general, the dimensions of the fibers making up such matrix materials will be in the range of about 1 μm to 1 mm, depending on the final conduit size, and on the required matrix flexibility. Any suitable matrix material may be utilized so long as it can be impregnated with a suitable resin, formed into an artificial conduit and function as described herein. In a preferred embodiment, the matrix for a stent will be polyester, and the matrix for a vascular graft will be woven polyester.

The exact composition of the composite used to form a tailored conduit will vary from situation to situation. However, in general the resin-matrix composite will be from about 20% to about 50% by weight matrix fabric.

In one embodiment of the present invention, the tailored, biocompatible conduits of the present invention may be utilized as drug delivery devices. In this embodiment, the conduits may have a dual function, i.e. that of providing a conduit as described herein and the delivery of pharmacologically active agents. For example, the resins may be impregnated with therapeutic agents which serve to enhance healing of the vessel, inhibit the formation of scar tissue, prevent clot formation, and the like. Examples of materials which may be utilized in this manner include but are not limited to antibiotics, anti-clotting agents, anti-inflammatory agents, growth factors, chemotactic agents, and the like. However, the conduits may serve as drug delivery devices without necessarily performing a structural function for a vessel. For example, the composite may be impregnated with a pharmacologically active agent and positioned at a location of interest for the sole purpose for delivering the agent. In particular, the composition of the composite may be designed in order to effect a “timed release” type of delivery in that the active agent is released gradually over a period of time. Such a design may be accomplished by altering parameters such as the porosity of the resin to allow an active agent to diffuse out of the conduit. Alternatively, the agent may be associated with the conduit via a labile linkage that is susceptible to dissolution over time by conditions in the environment at the site of placement of the conduit, e.g. by pH, hydrolytic or enzymatic cleavage, etc. The active agent may be associated with the composite by any of various techniques known to those of skill in the art, e.g. by mixing with and permeating the resin and/or the matrix material with the agent prior to curing, or by attaching an active agent to the external surface of a cured composite e.g. by linking the agent to functional groups exposed on or near the surface of the conduit. Examples of pharmacologically active agents that may be delivered by the conduits of the present invention include any type of medication susceptible to being associated with the conduit, for example synthetic or naturally occurring small molecule drugs, proteins, polypeptides, chemotherapeutic agents, pain medications, as well as gene therapy agents (e.g. DNA, RNA, or vectors encoding DNA or RNA), and the like.

The tailored conduits of the present invention have a wide variety of applications. As stated above, one use for such a conduit is as a stent. By a “stent” we mean a medical device used in the tubular passage ways of the body to maintain an open lumen. Those of skill in the art will recognize that the conduits of the present invention may be utilized in many applications, including but not limited to intravascular stents (e.g. in the heart as in conjunction with angioplasty), airway stents, urologic stents, vetriculostomy tubes, bile duct stents, as surgical drains, and the like. The conduits of the present invention may be utilized in any medical application where the ability to place a narrow tube, expand the tube to a desired caliber and contour, and then solidify the tube, would be desirable.

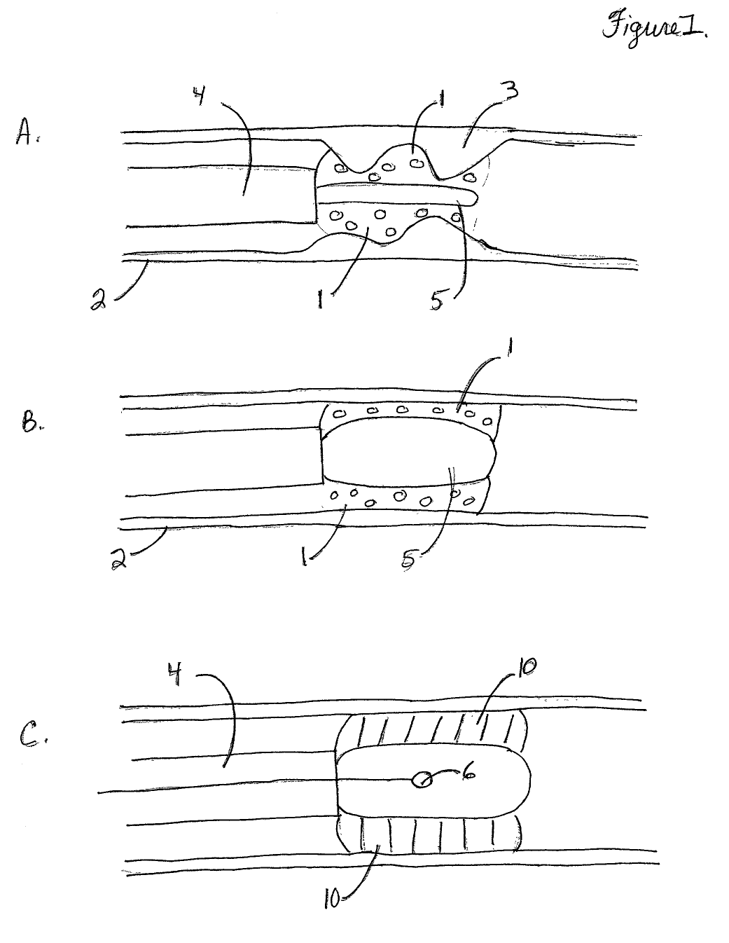

One embodiment of the invention (stent in conjunction with angioplasty) is illustrated in FIG. 1A-C. In this embodiment, a resin-impregnated matrix 1 is introduced into a vessel 2 at an occluded area to be stented 3 via a catheter 4 together with a balloon 5 that is deflated (1A). Upon inflation of the balloon 5, the occluded area 3 is opened and the resin-impregnated matrix 1 conforms to the shape of the vessel 2 (i.e. to the shape of the expanded previously occluded area, 1B). The cured resin-impregnated matrix 10 is set by exposure to visible light via the introduction of a light source 6 through the catheter 4. Upon removal of the balloon 5, the light source 6 and the catheter 4, the cured resin-impregnated matrix 10 remains to support the opened vessel. Procedures for the placement of conventional stents are well-known to those of skill in the art.

In another embodiment, the conduits of the present invention may function as tailored vascular grafts. In this embodiment, the resin-impregnated matrix may be, for example, in the form of a narrow sheet of material that can be “wrapped” around the junction between two vessels (e.g. to join two severed blood vessels) or placed in a manner so as to join a vessel and another structure such as an implanted artificial heart. The resin-impregnated matrix is positioned while in the uncured state and cured by exposure to visible light of a suitable wavelength. The resin can be subjected to a limited amount of light for a limited amount of time without polymerizing. This phenomenon is associated with the presence of a threshold in radiant energy below which not enough free radicals are created to initiate the resin polymerization reaction, allowing for a suitable working time for the resin in normal light. In addition, the wavelengths to which the resin is sensitive may be filtered out of the illumination source, extending the working time almost indefinitely.

The amount of resin-impregnated matrix that is utilized in a given application will vary from case to case, as will the exact dimensions (e.g. length and thickness) of the conduit that is formed.

The resins utilized in the practice of the present invention are preferably cured by exposure to wavelengths of light in the visible range, i.e. in the range of about 420 to about 700 nm. In a preferred embodiment of the present invention, the resin is cured by exposure to 470 nm light. Those of skill in the art will recognize that many suitable sources of light exist which may be used in the practice of the present invention, including but not limited to incandescent bulbs (mercury, tungsten, halide, etc.), lasers and light emitting diodes (LED). In a preferred embodiment of the present invention, GaN LEDs are used to cure the resin. They produce an illumination at 470 nm, which is close to the 468 nm maximum absorbance of camphorquinone. These light sources also offer the advantage of a small size and a good efficiency. For the cases where size is an issue, especially for the stent application, the illumination can be transported to the desired location by the mean of optic fibers.

One major strength of the present invention resides in the speed of hardening of the resin and of the whole resin-mesh composite. Only a very short illumination at the optimal wavelength is necessary to polymerize the resin, typically between about 10 to about 300 seconds. In addition, since the resin cure depends on the light dose, which is the product of the light intensity by the illumination time, high intensity lamps can lead to a further reduction in the illumination time necessary to obtain the full composite strength.

One inherent advantage of the conduits of the present invention is that their characteristics and properties may be varied almost infinitely according to a desired application. In addition to the composite size, thickness and shape, examples of parameters that may be varied include the elastic modulus (tensile strength),and the flexural modulus (flexibility) of the conduit.

The elastic modulus is an important property of the final cured resin-matrix composite. Those of skill in the art will recognize that the elastic modulus of a cured preparation may be purposefully varied by varying the resin composition and the matrix material, and that the desired elastic modulus may vary from application to application. However, in general, the desirable elastic modulus for the composite should be close to that of a natural arterial wall for the prosthetic application (about 100 kPa) and somewhat higher (superior to 100 kPa) for a stent application to be able to support the collapsed walls.

Likewise, the flexural modulus of a device fashioned from the resin-impregnated matrix of the present invention may be varied according to the composition of the resin and the mature of the matrix material. For example, the use of thick fiberglass versus thin fiberglass has shown to increase the flexural modulus by about 50%. Further, the addition of plasticizer to the resin formulation can increase the flexural modulus by nearly two-fold. While preferred flexural moduli may vary according to specific applications, in general the value should be close to that of a natural flexural modulus of a natural arterial wall, e.g. about 100 kPa.

### EXAMPLES

Fabrication of the Resin

The first step in mixing the resin consists in thinning bis-GMA, the principal monomer, (2 part by weight) with the viscosity modifier (1 part by weight). In the second step, 2% by weight of N, N dimethyl-p-toluidine is added. The third and last step, which transforms the resin into a photosensitive mix with the addition of 2% by weight of camphorquinone, needs to be performed in the dark or with controlled illumination, so that the wavelengths at which the photoinitiator is activated are eliminated. The resin can be stored at room temperature for an extended period of time, but under controlled light or in the dark.

Fabrication of the Resin-Impregnated Composite

In controlled lighting, a piece of mesh (nylon, fiberglass or other) was impregnated with the resin by simple dipping. The part was formed into the desired shape, flat in a “dog-bone” mold for the elastic modulus measurement, or as a 1.25-cm diameter cylinder on a mandrel for the flexural modulus measurement. Then, once everything was in position, the composite was created by illuminating the part for 1 or 2 minutes, causing the resin to polymerize. Lamps composed of an array of GaN LEDs illuminating in the visible blue were used.

Measurement of the Elastic and Flexural Moduli

The mechanical properties of the composite material were measured using a TA.XT2 Texture Analyzer (Texture Technology Corp., Scarsdale, N.Y.; Stable Micro Systems, Godalming Surrey, U.K.). Tensile grips were used to get the elastic modulus out of the dog-bone shaped samples. For the flexural modulus, the composite cylinders were tested in a three-point bending fixture.

### EXAMPLE 1

### Measurement of the Elastic Modulus

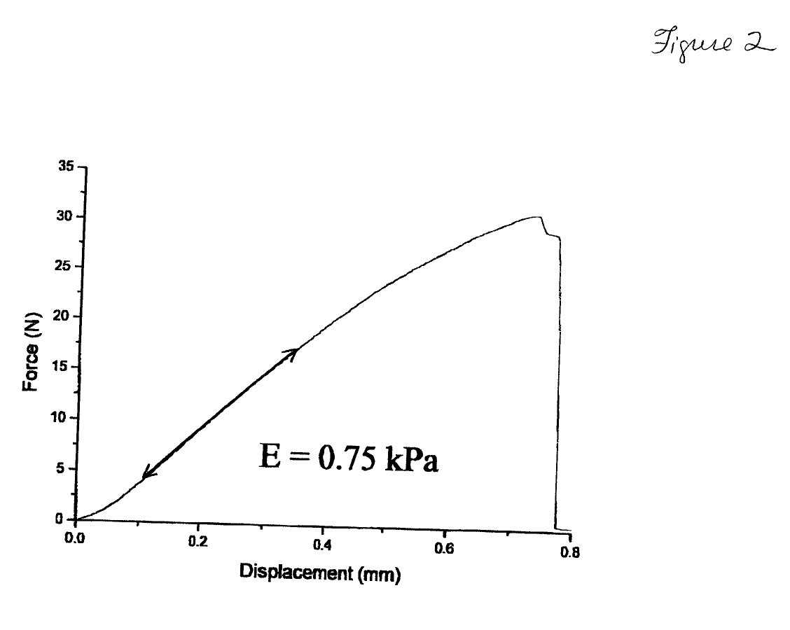

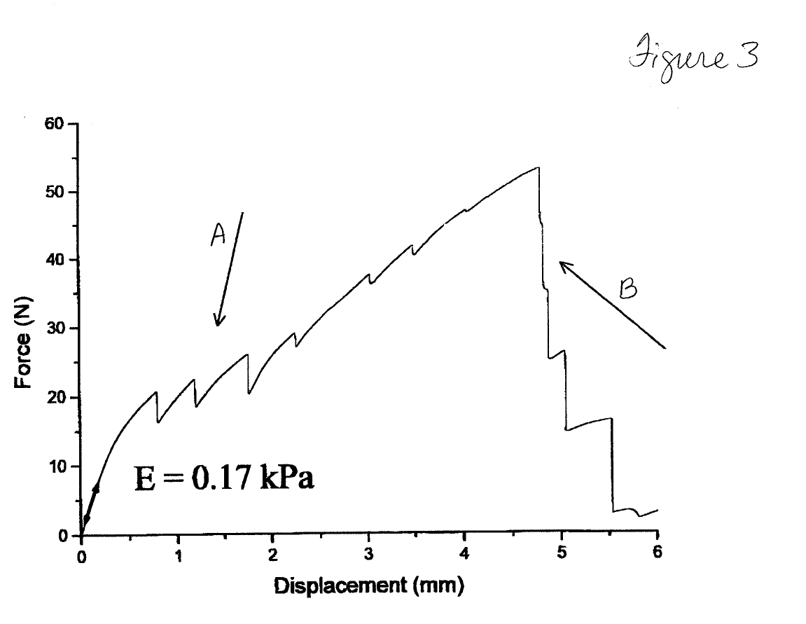

Dog-bone shaped resin/mesh samples were fabricated using a fiberglass fabric and a nylon fabric as described in Methods. The elastic modulus of the two materials was measured as described. Typical results for the thin fiberglass/basic resin composite (1 minute cure) and for the nylon/basic resin composite (2 minute cure) are shown in FIGS. 2 and 3, respectively. As can be seen, the nature of the mesh material has a dramatic effect on the mechanical behavior of the resulting composite.

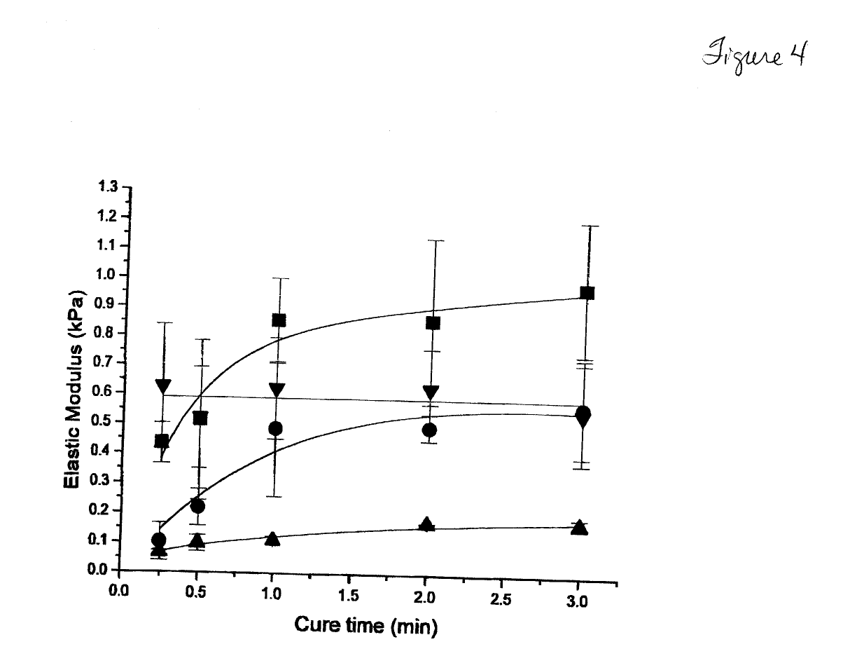

FIG. 4 shows a comparison of the effect of cure time on elastic modulus data when varying the mesh material (fiberglass vs. nylon), the mesh size (thick fiberglass vs. thick fiberglass) and with a plasticizer added to the resin formulation. As can be seen, many parameters of the fabrication of the mesh/resin composite have a strong influence on the elastic modulus of the final product. In addition, one can see that the elastic modulus values reach their plateau after only 1 to 2 minutes of exposure. The use of a higher intensity lamp would further reduce the necessary time.

This example demonstrates that, by varying fabrication parameters, light-cured composites can be made with characteristics (in particular, with elastic moduli) that render them suitable for use in the construction of tailored biocompatible conduits, such as stents and vascular prosthetic.

### EXAMPLE 2.

### Measurement of the Flexural Modulus

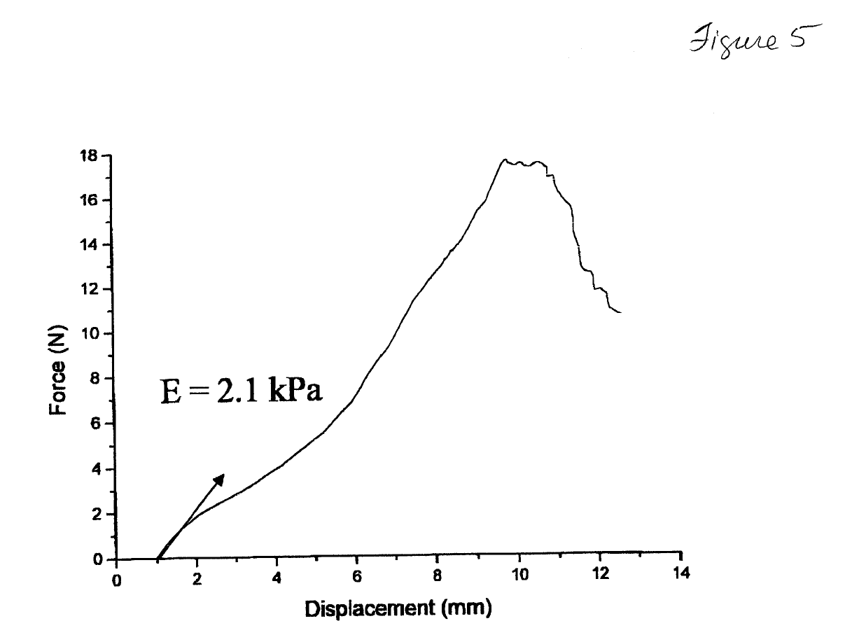

Composites in the form of tubes were fabricated as described in Methods using thin and thick fiberglass mesh and basic and plasticised resin. Their flexural modulus was measured using a threepoint bending fixture. A typical curve obtained for the basic resin/thin fiberglass composite (1 minute cure) is depicted in FIG. 5. As can be seen, the cylinder can withstand a deformation almost equal to its diameter before fracturing.

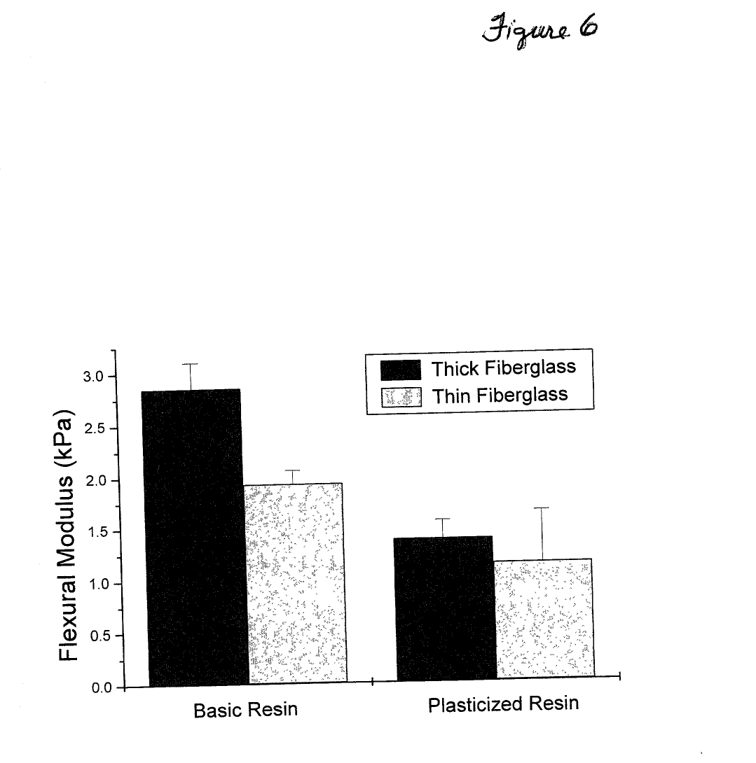

The flexural modulus of each type of tube was assessed as described and the results are depicted in FIG. 6. As can be seen, the size of the mesh as well as the composition of the resin have a strong effect on the flexural properties of the composite tubes.

This example demonstrates that, by varying fabrication parameters, light-cured composites can be made with characteristics (in particular, with flexural moduli) that render them suitable for use in the construction of tailored biocompatible conduits, such as stents and vascular prosthetic.

### EXAMPLE 3.

### Measurement of Adhesion Strength for Bonds Assembled and Cured Underwater on Various Substrates

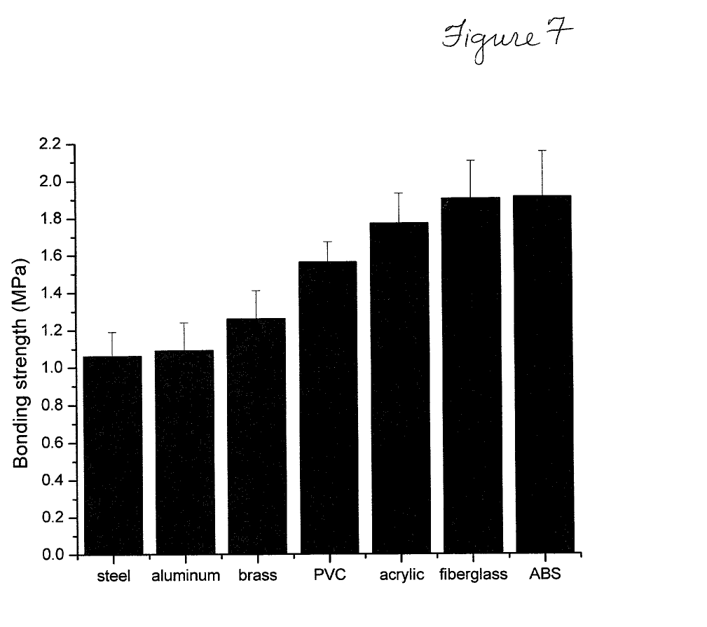

Bonds have been formed underwater using a variation of the basic resin (alkoxylated cyclohexane dimethanol diacrylate as viscosity modifier), between transparent cast acrylic as upper substrate and various metal (steel, aluminum, brass) and polymeric (PVC (polyvinyl chloride), acrylic, fiberglass, ABS (acrylonitrile/butadiene/styrene)) materials as the lower substrate. The substrate surfaces were lightly roughened with 100-grit sandpaper prior to bonding. The water was at room temperature and the resin was cured by a 1-minute illumination. The adhesive strength was measured in pure shear using a single-lap configuration and the TA.XT2 Texture Analyzer. The strength values are shown in FIG. 7.

This example demonstrates that the resin has the ability to cure and bond well in an aqueous environment such as that found in most physiological applications. This behavior should be preserved in the case of the fabrication of a resin/matrix composite. Since the human body is composed of a large quantity of various aqueous fluids, this property is of fundamental interest for applications involving in situ polymerization of the resin system.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### References

1. D. Cohn, Z. Elchai, B. Gershon, M. Karck, G, Lazarovici, J. Sela, M. Chandra, G. Marom, and G. Uretzky, J. Biomed. Mat. Res., 26 (9) 1185-1205, 1992.

2. N. L'Heureux, S. Paquet, R. Labbe, L. Germain, and F. A. Auger, FASEB J. 12 (1) 47-56, 1998.

3. J. H. Campbell, J. L. Efendy and G. R. Campbell. Circ. Res. 85 (12) 1173-1178, 1999.

4. C. Y. Park, J. K. Chang, D. Y. Jeong, G. J. Yoon, C. Chung, J. K. Kim D. C. Han and B. G. Min, ASAIO J. 43 (5) M647-M650, 1997.

5. R. C. Allen, r. A., White, C. K. Zarins, and T. J. Fogarty, J. Endo. Surg. 4 (2) 195-202, 1997.

6. H. Kasegawa, S. Kamato, T. Ida, M. Kawase, T. Fujimoto and M. Umezu, J. Heart Valve Dis. 6 (6) 604-607, 1997.

We claim:

1. A tailored artificial conduit comprising,
matrix material impregnated with a polymeric resin, wherein said polymeric resin is cured by exposure to radiant energy.

2. The tailored artificial conduit of claim 1, wherein said tailored artificial conduit is a stent.

3. The tailored artificial conduit of claim 1, wherein said tailored artificial conduit is a vascular prosthesis.

4. The tailored artificial conduit of claim 1 wherein said matrix material is selected from the group consisting of fiberglass, nylon, polyester, polyurethanes, polytetrafluoroethylene, cotton and silk.

5. The tailored artificial conduit of claim 1 wherein said light-cured polymeric resin comprises a principal monomer, a viscosity modifier, and a photoinitiator.

6. The tailored artificial conduit of claim 5 further comprising an activator.

7. The tailored artificial conduit of claim 5 wherein said principal monomer is selected from the group consisting of bis-phenol A diglycidyl methacrylate and acrylate monomers.

8. The tailored artificial conduit of claim 5 wherein said viscosity modifier is selected from the group consisting of triethylene glycol dimethacrylate, alkoxylated cyclohexane dimethanol diacrylate and difunctional monomers.

9. The tailored artificial conduit of claim 5 wherein said photoinitiator is selected from the group consisting of camphorquinone, ketones, thioxanthone and 3-ketocoumarins.

10. The tailored artificial conduit of claim 6 wherein said activator is selected from the group consisting of N,N dimethyl-p-toluidine, amines, and tertiary amines.

11. The tailored artificial conduit of claim 1 wherein said radiant energy is 470 nanometers in wavelength.

12. The tailored artificial conduit of claim 1 further comprising a biologically active agent.

13. The tailored artificial conduit of claim 12 wherein said biologically active agent is selected from the group consisting of antibiotics, anti-rejection drugs, anti-coagulants, anti-inflammatory agents, growth factors, and chemotactic agents.

14. A method of fabricating a tailored artificial conduit comprising the steps of
impregnating a matrix with uncured photoactivatable resin in order to form impregnated matrix material, wherein said photoactivatable resin is susceptible to curing by exposure to visible light,
positioning said impregnated matrix material at a physiological site of interest, and
exposing said impregnated matrix material to radiant energy, wherein said step of exposing cures said uncured photoactivatable resin within said matrix, thereby forming a tailored artificial conduit.

15. The method of claim 14 further comprising the step of forming said impregnated matrix material into a conduit at said site of interest, wherein said conduit conforms to the natural shape of said site of interest.

16. The method of claim 14 wherein said tailored artificial conduit functions as a stent.

17. The method of claim 14 wherein said tailored artificial conduit functions as a vascular prosthesis.

18. The method of claim 14 wherein said matrix is selected from the group consisting of fiberglass, nylon, polyester, polyurethane, polytetrafluoroethylene, cotton and silk.

19. The method of claim 14 wherein said uncured photoactivatable resin comprises a principal monomer, a viscosity modifier, and a photoinitiator.

20. The method of claim 19 further comprising an activator.

21. The method of claim 19 wherein said principal monomer is selected from the group consisting of bis-phenol A diglycidyl methacrylate and acrylate monomers.

22. The method of claim 19 wherein said viscosity modifier is selected from the group consisting of triethylene glycol dimethacrylate, alkoxylated cyclohexane dimethanol diacrylate and difunctional monomers.

23. The method of claim 19 wherein said photoinitiator is selected from the group consisting of camphorquinone, ketones, thioxanthone and 3-ketocoumarins.

24. The method of claim 20 wherein said activator is selected from the group consisting of N,N dimethyl-p-toluidine, amines and tertiary amines.

25. The method of claim 14 wherein said radiant energy is of a 470 nm wavelength.
